SEQUENCE LISTING (8 sequences)

SEQ ID NO: 1 - DNA, 91 bp, Artificial Sequence
  It is synthesized
ccgctcgaggccaccatggattacaaggatgacgacgataagtgtaagggaggaagagcgaaggattgtgcggtggaaggaggaatgaaat

SEQ ID NO: 2 - DNA, 33 bp, Artificial Sequence
  It is synthesized.
ccggaattcctacatcacctcgtagccacttct

SEQ ID NO: 3 - DNA, 768 bp, Artificial Sequence
  It is synthesized.
atggattacaaggatgacgacgataagtgtaagggaggaagagcgaaggattgtgcggtggaaggaggaatgaaatgtgtgaagttcttgctctacgtcctcctgctggccttttgcgcctgtgcagtgggactgattgccgtgggtgtcggggcacagcttgtcctgagtcagaccataatccagggggctacccctggctctctgttgccagtggtcatcatcgcagtgggtgtcttcctcttcctggtggcttttgtgggctgctgcggggcctgcaaggagaactattgtcttatgatcacgtttgccatctttctgtctcttatcatgttggtggaggtggccgcagccattgctggctatgtgtttagagataaggtgatgtcagagtttaataacaacttccggcagcagatggagaattacccgaaaaacaaccacactgcttcgatcctggacaggatgcaggcagattttaagtgctgtggggctgctaactacacagattgggagaaaatcccttccatgtcgaagaaccgagtccccgactcctgctgcattaatgttactgtgggctgtgggattaatttcaacgagaaggcgatccataaggagggctgtgtggagaagattgggggctggctgaggaaaaatgtgctggtggtagctgcagcagcccttggaattgcttttgtcgaggttttgggaattgtctttgcctgctgcctcgtgaagagtatcagaagtggctacgaggtgatgtag

SEQ ID NO: 4 - DNA, 927 bp, Artificial Sequence
  It is synthesized.
ggccggggggcgcagctagagagccccggagccgcggcgggagaggaacgcgcagccagccttgggaagcccaggcccggcagccatggcggtggaaggaggaatgaaatgtgtgaagttcttgctctacgtcctcctgctggccttttgcgcctgtgcagtgggactgattgccgtgggtgtcggggcacagcttgtcctgagtcagaccataatccagggggctacccctggctctctgttgccagtggtcatcatcgcagtgggtgtcttcctcttcctggtggcttttgtgggctgctgcggggcctgcaaggagaactattgtcttatgatcacgtttgccatctttctgtctcttatcatgttggtggaggtggccgcagccattgctggctatgtgtttagagataaggtgatgtcagagtttaataacaacttccggcagcagatggagaattacccgaaaaacaaccacactgcttcgatcctggacaggatgcaggcagattttaagtgctgtggggctgctaactacacagattgggagaaaatcccttccatgtcgaagaaccgagtccccgactcctgctgcattaatgttactgtgggctgtgggattaatttcaacgagaaggcgatccataaggagggctgtgtggagaagattgggggctggctgaggaaaaatgtgctggtggtagctgcagcagcccttggaattgcttttgtcgaggttttgggaattgtctttgcctgctgcctcgtgaagagtatcagaagtggctacgaggtgatgtaggggtctggtctcctcagcctcctcatctgggggagtggaatagtatcctccaggtttttcaattaaacggattattttttcaaaaaaaaaaaaaaaaaaaaaaaaaaaaaaaaaaaaaaaaaaaa

SEQ ID NO: 5 - DNA, 714 bp, Artificial Sequence
  It is synthesized.
atggcggtggaaggaggaatgaaatgtgtgaagttcttgctctacgtcctcctgctggccttttgcgcctgtgcagtgggactgattgccgtgggtgtcggggcacagcttgtcctgagtcagaccataatccagggggctacccctggctctctgttgccagtggtcatcatcgcagtgggtgtcttcctcttcctggtggcttttgtgggctgctgcggggcctgcaaggagaactattgtcttatgatcacgtttgccatctttctgtctcttatcatgttggtggaggtggccgcagccattgctggctatgtgtttagagataaggtgatgtcagagtttaataacaacttccggcagcagatggagaattacccgaaaaacaaccacactgcttcgatcctggacaggatgcaggcagattttaagtgctgtggggctgctaactacacagattgggagaaaatcccttccatgtcgaagaaccgagtccccgactcctgctgcattgatgttactgtgggctgtgggattaatttcaacgagaaggcgatccataaggagggctgtgtggagaagattgggggctggctgaggaaaaatgtgctggtggtagctgcagcagcccttggaattgcttttgtcgaggttttgggaattgtctttgcctgctgcctcgtgaagagtatcagaagtggctacgaggtgatg

SEQ ID NO: 6 - DNA, 24 bp, Artificial Sequence
  It is synthesized.
gattacaaggatgacgacgataag

SEQ ID NO: 7 - DNA, 27 bp, Artificial Sequence
  It is synthesized.
tgtaagggaggaagagcgaaggattgt

SEQ ID NO: 8 - protein, 9 aa, Artificial Sequence
  It is synthesized
CKGGRAKDC

(19) United States
(12) Patent Application Publication
(10) Pub. No.: US 2019/0133964 A1
(43) Pub. Date: May 9, 2019

(54) Polypeptide nano-bubbles and preparation method and application thereof

(71) Applicant: NANJING MEDICAL UNIVERSITY, Nanjing (CN)

(72) Inventors: Yaqin ZHANG, Nanjing (CN); Xiao HAN, Nanjing (CN); Yi YUAN, Nanjing (CN); Hongwei LI, Nanjing (CN); Xiaoai CHANG, Nanjing (CN); Jing PANG, Nanjing (CN); Jingjing WANG, Nanjing (CN); Bin QIAN, Nanjing (CN); Heming WU, Nanjing (CN); Tingting YU, Nanjing (CN); Jindao WU, Nanjing (CN); Ningyuan TANG, Nanjing (CN); Liyong PU, Nanjing (CN); Rufeng XU, Nanjing (CN)

(21) Appl. No.: 16/232,770

(22) Filed: Dec. 26, 2018

(30) Foreign Application Priority Data
Jun. 6, 2018 (CN) 2018105736289

(51) Int. Cl. A61K 9/51; A61K 47/69; A61K 31/12; A61P 3/04; C07K 14/705; C07K 14/47

(52) U.S. Cl. CPC A61K 9/5192; A61K 47/6925; A61K 31/12; A61P 3/04; C07K 2319/60; C07K 14/70596; C07K 14/47; C07K 2319/43; A61K 9/5169

(57) ABSTRACT

A preparation method for polypeptide nano-bubbles, comprising the following steps: constructing a recombinant plasmid, which includes a Flag tag, an adipose tissue-targeting polypeptide and the coding gene of nano-bubble marker membrane protein CD63; and transfecting the recombinant plasmid into cells which secrete nano-bubbles through lipidosome for culturing, collecting a cell culture solution, and extracting the polypeptide nano-bubbles by ultracentrifugation. The present invention further discloses the polypeptide nano-bubbles and application thereof in the preparation of drugs for treating obesity. The polypeptide nano-bubbles bring great convenience for targeted therapy of anti-obesity drugs. The polypeptide nano-bubbles have good biocompatibility and are capable of carrying different kinds of bioactive substances.

This application claims priority to Chinese Patent Application Ser. No CN2018105736289 filed on 6 Jun. 2018.

### TECHNICAL FIELD

The present invention relates to nano-bubbles and application thereof, and in particular to polypeptide nano-bubbles and a preparation method and application thereof.

### BACKGROUND ART

With the improvement of people's living standard, obese people are increasing year by year. The World Health Organization reports that, as a chronic metabolic disease, obesity has become one of the five major risk factors for death worldwide. The reason is that obesity may cause various metabolic diseases, such as type 2 diabetes, cardiovascular and cerebrovascular diseases and tumors. Therefore, the intervention therapy for obesity is an important measure to prevent and treat obesity-related diseases.

So far, the treatment of obesity mainly comprises dietary behavior therapy, surgeries and drug therapy. For the dietary behavior therapy, when you stop losing weight, the weight will be regained severely, and even exceeds the weight before losing weight. Risks of the surgeries (such as sleeve gastrectomy, gastric bypass and gastric banding) are too high. Therefore, numerous obese patients take drug therapy as the first choice. At present, two anti-obesity drugs authorized by United States FDA are sibutramine and orlistat. But the two anti-obesity drugs have large side effects after being orally taken for a long time. For example, the sibutramine may cause high blood pressure, insomnia, etc., and the orlistat may cause diarrhea, acathexia, etc,. Therefore, how to make drugs only target adipose tissue instead of other internal organs and tissues in metabolic syndromes-related organs so as to achieve the goal of efficiently treating metabolic syndromes while not changing the human immune system has become an important issue and research focus in the treatment of obesity.

In 2004, the well-known scientific journal “Nature Medicine” reported that a polypeptide having an amino acid sequence of CKGGRAKDC, could bind with prohibitin (endothelial cell receptors) in white adipose tissue vessels, and thus can target white adipose tissue vessels. To test whether the polypeptide can carry materials having pharmacological effects and bioactivity targeting the white adipose tissue vessels, angiogenesis is inhibited through destruction of structures of the adipose tissue vessels, thereby inhibiting obesity due to accumulation of adipose. The targeting polypeptide is connected to (KLAKLAK)₂ through glycinylglycine bridge to form CKGGRAKDC-GG-_{D}(KLAKLAK)₂ of targeted adipose tissue vessels having 25 residues. However, CKGGRAKDC-GG-_{D}(KLAKLAK)₂ formed by connecting the targeting polypeptide and (KLAKLAK)₂ limits drug loading of the targeting polypeptide. Specifically, in terms of the structural formula of the targeting polypeptide, one molecule of the targeting polypeptide can only carry one molecule of (KLAKLAK)₂ to enter into adipose tissues, so that higher injection dose is required for the same drug effect, thereby increasing therapy costs. Thus, the effect of the prior art technologies for treatment of obesity is limited, thereby limiting the general usage of targeting drugs for treatment of obesity. Further, variety of drugs carried by the targeting polypeptide and biological safety of polypeptide-drug compound are key pending issues during treatment of obesity. Thus, in terms of drawbacks in the prior art, there is a desire to develop a new targeting carrier carrying drugs for safe and effective treatment of obesity.

### SUMMARY OF THE INVENTION

OBJECTIVE: To develop a targeting carrier which can carry various drugs and have a high drug loading, the first aspect of the present invention provides a preparation method of polypeptide nano-bubbles, the second aspect of the present invention provides polypeptide nano-bubbles prepared by the preparation method, and the third aspect of the present invention provides application of the polypeptide nano-bubbles. Polypeptide nano-bubbles are used for targeted therapy of obesity by encapsulating drugs (such as drugs of nucleic acids, proteins, lipids, carbohydrates, ketones, etc.) and different ways of administration (such as intravenous injection, subcutaneous or intraperitoneal injection, etc.).

TECHNICAL SCHEME: The first aspect of the present invention provides a preparation method of the polypeptide nano-bubbles, comprising the following steps:
(1) constructing a recombinant plasmid, the recombinant plasmid comprising coding genes of a Flag tag, an adipose tissue-targeting polypeptide and a micro-bubble marker membrane protein CD63, wherein the coding genes of the Flag tag and the adipose tissue-targeting polypeptide are successively linked to the rear of a start codon of the coding gene of the micro-bubble marker membrane protein CD63, and the amino acid sequence of the adipose tissue-targeting polypeptide is CKGGRAKDC; and
(2) transfecting the recombinant plasmid obtained in step (1) into cells which secrete nano-bubbles through lipidosome, culturing to collect a cell culture solution and extracting the polypeptide nano-bubbles by ultracentrifugation.

In step (1), steps of construction of the recombinant plasmid are as follows: amplifying a Flag-peptide-CD63 fragment by PCR using cDNA of CD63 as the template and CD63-F/CD63-R as the primers, and inserting the Flag-peptide-CD63 fragment into XhoI and EcoRI digestion sites of the carrier plasmid pIRES2-EGFP, wherein the nucleotide sequence of the primer CD63-F is shown in SEQ ID NO: 1, and the nucleotide sequence of the primer CD63-R is shown in SEQ ID NO: 2; and the nucleotide sequence of the Flag-peptide-CD63 fragment is shown in SEQ ID NO: 3.

The nucleotide sequence of cDNA of CD63 is shown in SEQ ID NO: 4, and comprises upstream 5′ non-coding sequence+ CDS+ downstream 3′ non-coding sequence, and the nucleotide sequence of CDS is shown in SEQ ID NO: 5. Preferably, pOTB7-CD63 is used as a template.

CD63-F contains an XhoI digestion site (CTCGAG), a Flag tag sequence (GATTACAAGGATGACGACGATAAG) and an adipose tissue-targeting polypeptide CKGGRAKDC sequence (TGTAAGGGAGGAAGAGCGAAGGATTGT); and CD63-R contains an EcoRI digestion site (GAATTC).

Definitions of amino acid residues represented by various alphabetic symbols in the amino acid sequence of the adipose tissue-targeting polypeptide are as follows: C is aminothiopropionic acid, K is lysine, G is glycine, R is arginine, A is alanine, and D is aspartic acid.

In step (2), the cells which secrete nano-bubbles can be immature dendritic cells, mesenchymal stem cells and other cells (such as 293T cells), and the 293T cells are taken as an example for illustration herein. The recombinant plasmids are guided into the 293T cells according to an instruction of a lipofectamine®3000 transfection reagent (article number: L3000015); and steps of ultracentrifugation are as follows: centrifuging the cell culture solution at 500-1000 g, 2-4° C. for 10-30 min, then taking first-time supernatant, and removing cell precipitates; centrifuging the first-time supernatant at 1500-3000 g, 2-4° C. for 20-30 min, then taking second-time supernatant, and removing some cell debris or residual cell organelle precipitates; and centrifuging the second-time supernatant at 100,000-160,000 g, 2-4° C. for 1-2 h to obtain precipitates, namely the polypeptide nano-bubbles.

The second aspect of the invention provides the polypeptide nano-bubbles prepared by the preparation method, and the nano-bubbles are polypeptide nano-bubbles modified by adipose tissue-targeting polypeptides.

The third aspect of the invention provides the application of the polypeptide nano-bubbles as a drug carrier in preparation of drugs for treating obesity.

Preferably, the polypeptide nano-bubbles carry the drugs for treating obesity in an encapsulating manner, and the drugs for treating obesity are nucleic acids drugs, proteins drugs, lipids drugs, carbohydrates drugs or ketones drugs. According to difference of the carried drugs, a polypeptide nano-bubble-drug preparation is prepared by different methods.

More preferably, the ketones drugs are curcumin. The curcumin is mixed with the polypeptide nano-bubbles, the mixture is cultured at a room temperature, and then 200,000 g of the mixture is centrifuged for 1.5-3 h via sucrose density gradient (the concentrations of sucrose are separately 10 wt. %, 20 wt. %, 30 wt. %, 40 wt. %, 50 wt. %, 60 wt. % and 70 wt. %), and the mixture is collected on a yellow band in the middle of 40-60% sucrose concentration, and is subjected to PBS resuspension to obtain a polypeptide nano-bubble-curcumin preparation, the polypeptide nano-bubbles encapsulate the curcumin, thus, the stability of the curcumin is improved, and the toxicity of the curcumin is reduced.

The invention has the following beneficial effects: (1) the invention provides the new polypeptide nano-bubbles, which provide great convenience for targeted treatment of anti-obesity drugs. The polypeptide nano-bubbles can carry different kinds of bioactive substances, and also have good biocompatibility. The encapsulation of the carried drugs by the polypeptide nano-bubbles makes the drugs have good biological compatibility. Meanwhile, the stability of the drugs in vivo is obviously enhanced, the circulation time of the drugs in vivo is prolonged, and the utilization rate of the drugs is greatly improved, thus significantly enhancing the targeting anti-obesity activity of the drugs. The polypeptide nano-bubbles are suitable for different drug delivery methods. For example, the polypeptide nano-bubbles may function as help targeting in intravenous injection and sustained releasing in intraperitoneal, intramuscular and subcutaneous injections. (2) The preparation method of the patent is simple in process, and properties of the drugs are not damaged; and the prepared polypeptide nano-bubbles are stable in property, according to the preparation method, targeting polypeptides are linked to nano-bubble membrane protein by molecular cloning and cell transfection, the structures and properties of the polypeptide nano-bubbles obtained by the modifying method are quite stable, target-missing or degradation caused by change of an external environment is avoided, and compared with artificially synthesized polypeptide chemically modified nano-bubbles, the polypeptide nano-bubbles prepared by the preparation method have remarkable advantages, and are favorably stored for a long time.

### BRIEF DESCRIPTION OF THE DRAWINGS

FIG. 1 is a plasmid profile of pIRES2-EGFP;

FIG. 2 is a schematic diagram of recombinant plasmid of pIRES2-EGFP-Flag-peptide-CD63;

FIG. 3 is a schematic diagram of construction and drug carrying of polypeptide nano-bubbles;

FIG. 4 is characterization analysis to the polypeptide nano-bubbles and a polypeptide nano-bubble-curcumin preparation by TEM and NTA separately, an upper figure is a photo shot by TEM, and a lower figure is a detection analysis result of NTA.

FIG. 5 is change of weight of mice fed with high-fat diet before and after drug administration; ^{$$}P<0.01 and ^{$$$}P<0.001 show comparison with a normal control group; *P<0.05, **P<0.01 and ***P<0.001 show comparison with a model control group; ^{#}P<0.05 and ^{##}P<0.01 show comparison with a curcumin group; and ^{&}P<0.05 and ^{&&}P<0.01 show comparison with a nano-bubble-curcumin group;

FIG. 6 is amounts of red fluorescence labeled nano-bubble-curcumin and polypeptide nano-bubble-curcumin which enter adipocytes under the observation of a laser scanning confocal microscope; and

FIG. 7 shows detection of pathological change of livers and kidneys of mice in various groups by H-E staining.

### DETAILED DESCRIPTION OF THE INVENTION

### Embodiment 1 Preparation of Polypeptide Nano-Bubbles and a Polypeptide Nano-Bubble-Curcumin Preparation

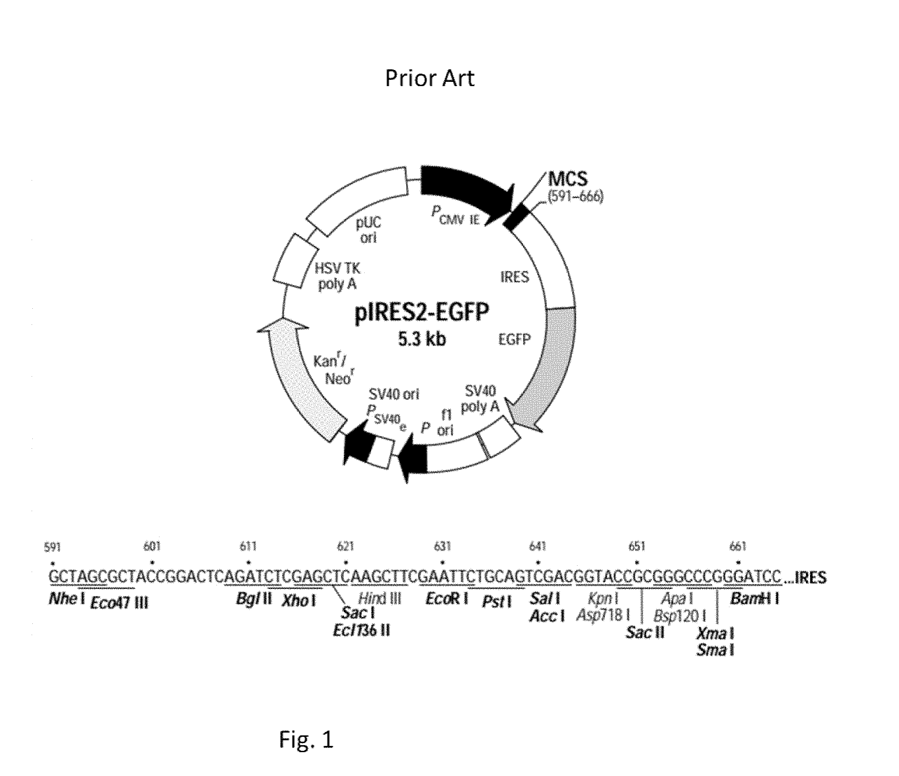

Experimental plasmid: carrier plasmid which is purchased by a company and contains an EGFP reporter gene is pIRES2-EGFP, and a map thereof is shown in FIG. 1.

Experimental Steps:
1) recombinant plasmid which contains a Flag tag, a targeting polypeptide CKGGRAKDC and the CD63 gene is constructed by taking pIRES2-EGFP as a carrier; and detailed steps are as follows:
   1.1 Primer design
      according to a CDS sequence of CD63 and an MCS site of a pIRES2-EGFP expression vector, a specific PCR primer is designed: the nucleotide sequence of a primer CD63-F is shown in SEQ ID NO: 1, and the nucleotide sequence of a primer CD63-R is shown in SEQ ID NO: 2, wherein CD63-F contains an XhoI digestion site (CTCGAG), a Flag tag sequence (GATTACAAGGATGACGACGATAAG) and an adipose tissue-targeting polypeptide CKGGRAKDC sequence (TGTAAGGGAGGAAGAGCGAAGGATTGT); and CD63-R contains an EcoRI digestion site (GAATTC).
   1.2 PCR reaction
      A following reaction system is established in a 50 μL PCR reaction tube, shown in Table 1:

TABLE 1
PCR system of Flag-peptide-CD63

| Component | Manufacturer | Volume |
|---|---|---|
| 5xPfu Reaction | TakaRa (PC1300) | 10 μL |
| Buffer | | |
| pOTB7-CD63 | Nanjing Jingmai Biotech Co., Ltd | 1 μL (10 ng) |
| | (PPL00300) | |
| CD63-F (20 mM) | Shanghai Generay Gene Co., Ltd. | 1.5 μL |
| CD63-R (20 mM) | Shanghai Generay Gene Co., Ltd. | 1.5 μL |
| dNTP (2.5 mM) | TakaRa (4030) | 4 μL |
| Pfu Polymerase | TakaRa (PC1300) | 1 μL (1 U) |
| | | Supplement |
| | | H2O to 50 μL |

The cDNA sequence of CD63 in pOTB7-CD63 is shown in SEQ ID NO: 4, and comprises upstream 5′ non-coding sequence+ CDS+ downstream 3′ non-coding sequence, and a nucleotide sequence of CDS is shown in SEQ ID NO: 5.
Reaction conditions are as follows:

$$94 ° C . 5 min \\ \begin{matrix}94 ° C . 15 s \\ 58 ° C . 15 s \\ 72 ° C . 50 s\end{matrix} \} 24 cycles$$

$$72 ° C . 10 min$$

Cooling to 4° C. is carried out, and after reaction is finished, 10 μL is taken for 1.0% agarose electrophoresis identification.
1.3 Digestion and connection of DNA
   Digestion and connection of DNA are carried out according to a conventional molecular cloning method, and specific steps are as follows:
      1.3.1 After a PCR product obtained in step 1.2 is subjected to electrophoretic separation, a target fragment is recycled by gel cutting, and a digestion reaction system shown in Table 2 is established:

TABLE 2
Digestion reaction system of Flag-peptide-CD63 fragment

| Component | Manufacturer | Volume |
|---|---|---|
| Fragment | | 16 μL (1 μg) |
| 10x Buffer H | TakaRa (1094S) | 2 μL |
| XhoI | TakaRa (1094S) | 1 μL (15 U) |
| EcoRI | TakaRa (1040S) | 1 μL (15 U) |

A digestion reaction system shown in Table 3 is established for the pIRES2-EGFP carrier:

TABLE 3
Digestion reaction system of pIRES2-EGFP carrier

| Component | Manufacturer | Volume |
|---|---|---|
| pIRES2-EGFP carrier | Clontech | 16 μL (1 μg) |
| 10x Buffer H | TakaRa (1094S) | 2 μL |
| XhoI | TakaRa (1094S) | 1 μL (15 U) |
| EcoRI | TakaRa (1040S) | 1 μL (15 U) |

After all the digestion reaction systems react overnight at 37° C., target fragments are recycled by gel cutting.
1.3.2 After DNA fragments obtained after digestion is carried out are purified via gel cutting, a connection reaction system shown in Table 4 is established:

TABLE 4
Construction of connection reaction system of recombinant plasmids

| Component | Manufacturer | Volume |
|---|---|---|
| 10x Ligation Buffer | TakaRa (9015-85-4) | 2 μL |
| Flag-peptide-CD63 fragments | | 12 μL (0.2 pmol) |
| obtained after digestion | | |
| Non-load of pIRES2-EGFP | | 3 μL (0.03 pmol) |
| obtained after digestion | | |
| PEG4000 (40 g/100 mL) | Shanghai Lianshuo | 2 μL |
| | Biological | |
| | Technology Co, | |
| | Ltd. (60365ES76) | |
| T4 DNA Ligase | TakaRa (9015-85-4) | 1 μL (350 U) |

and reaction is carried out for 16 h at 22° C.
1.4 Transformation of connection product
   1.4.1 20 μL connecting liquid obtained in step 1.3.2 is totally added in 100 μL TOP10 competent cells (Nanjing Saihongrui Biotech Co, Ltd.), and the mixture is placed on ice for 30 min;
   1.4.2 the mixture is subjected to heat shock for 90 s at 42° C., and is rapidly placed in ice and maintained for 5 min, and then 600 μL of the LB culture solution preheated at 37° C. is added;
   1.4.3 the mixture is shaken out for 30 min at 220 rpm and at 37° C., is centrifuged, and then totally coats the LB flat plate which contains 50 μg/mL Kan, and the LB flat plate is inverted for culturing overnight at 37° C., wherein
      a formula of the LB culture solution is as follows: when every liter of a culture medium is prepared, the following components are added in 950 mL of deionized water: 10 g of tryptone, 5 g of a yeast extract and 10 g of NaCl; a container is shaken until the components are dissolved completely, pH is regulated to 7.0 by using 5 mol/L of a NaOH aqueous solution, and then the volume is adjusted to 1 L with deionized water. Steam sterilization is carried out for 20 min under 15 psi high pressure, and the LB culture solution is stored at 4° C. for subsequent use.
      A formula of the LB flat plate is as follows: 1) preparation: 1 L of the LB culture medium (10 g/L of the tryptone, 5 g/L of the yeast extract and 10 g/L of NaCl) is taken, and 15 g of agar powder is added; 2) adding of antibiotic: after high-pressure sterilization is carried out, the melted LB solid culture medium is placed in water bath at 55° C., antibiotic is added after the temperature of the culture medium is reduced to 55° C. (people can touch the culture medium with hands) so as to prevent the antibiotic from losing efficacy due to over-high temperature, and the mixture is shaken up fully; 3) plate inverting: a plate is inverted at 10 mL generally. After the culture medium is poured into a culture dish, a cover is opened, and the culture medium is illuminated for 10-15 min under ultraviolet rays; and 4) storing: the edge of the culture dish is sealed with a sealing adhesive, and the culture dish is inverted and stored at 4° C., wherein the LB flat plate needs to be used within one month.
1.5 Positive clone identification
   4 monoclones are randomly selected and added into a test tube which contains 50 μg/mL Kan of 4 mL LB culture solution, the test tube is shaken out for 4 h at 220 rpm at 37° C., 100 μL of the 4 mL LB culture solution is taken and centrifuged, thallus precipitates are taken, are re-suspended with 50 μL ddH2O, are subjected to water bath for 5 min with boiling water, and are centrifuged, and 1 μL supernate is taken as a template for PCR identification; and a 50 μL PCR reaction system is shown in Table 5.

TABLE 5
Bacterial colony PCR reaction system

| Component | Manufacturer | Volume |
|---|---|---|
| 2xTaq Master Mix | TakaRa (D337A) | 25 μL |
| Supernate | | 1 μL |
| CD63-F (20 mM) | Shanghai Generay | 1 μL |
| | Gene Co., Ltd. | |
| CD63-R (20 mM) | Shanghai Generay | 1 μL |
| | Gene Co., Ltd. | Supplement H2O to |
| | | 50 μL |

Reaction conditions are as follows:

$$94 ° C . 10 min \\ \begin{matrix}94 ° C . 30 s \\ 58 ° C . 30 s \\ 72 ° C . 50 s\end{matrix} \} 25 cycles$$

$$72 ° C . 10 min$$

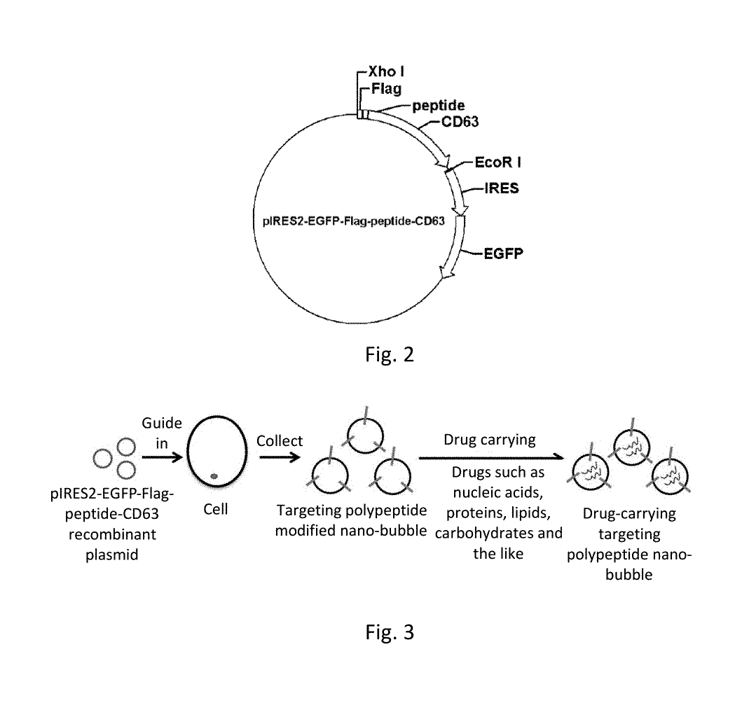

Cooling to 4° C. is carried out, and after reaction is finished, 10 μL is taken for 1.0% agarose electrophoresis identification.
1.6 DNA sequencing
   Components identified as positive clones via bacterial colony PCR are sequenced, and the nucleotide sequence of Flag-peptide-CD63 is shown in SEQ ID NO: 3. A constructed recombinant plasmid structure diagram is shown in FIG. 2.
2) Constructed recombinant plasmids DNA is transfected into source cells 293T which secrete micro-bubbles by lipidosome.
   According to an instruction of a lipofectamine®3000 transfecting reagent (article number: L3000015) of the invitrogen company, the recombinant plasmids DNA is guided into 293T cells. Specific steps are as follows: the 293T cells (ATCC, product number: CM-H010) are inoculated into a cell culture solution, the cell culture solution is cultured in a 5% CO2 culture tank at 37° C., and is transfected when the density reaches 70-90% (about 0.25-1×10⁶ cells); a 3.76-7.5 μL lipofectamine®3000 reagent is diluted with a 125 μL Opti-MEM culture medium, and the 3.76-7.5 μL lipofectamine®3000 reagent and the 125 μL Opti-MEM culture medium are fully mixed uniformly; recombinant plasmids 5 μg DNA (with the concentration being 1-5 μg/μL) is diluted with a 250 μL Opti-MEM culture medium to prepare DNA premix, then 10 μL P3000™ reagent (2 μL/μg DNA) is added, and the DNA premix and the 10 μL P3000™ reagent (2 μL/μg DNA) are fully mixed uniformly; diluted DNA (125 μL) is added in every tube of diluted ipofectamine®3000 reagent (125 μL) (at the ratio of 1:1), and the diluted DNA (125 μL) and the ipofectamine®3000 reagent (125 μL) are incubated for 5 min at the room temperature; and a lipidosome-DNA compound (250 μL) is added in cells, and the cells are incubated for 48 h at 37° C.

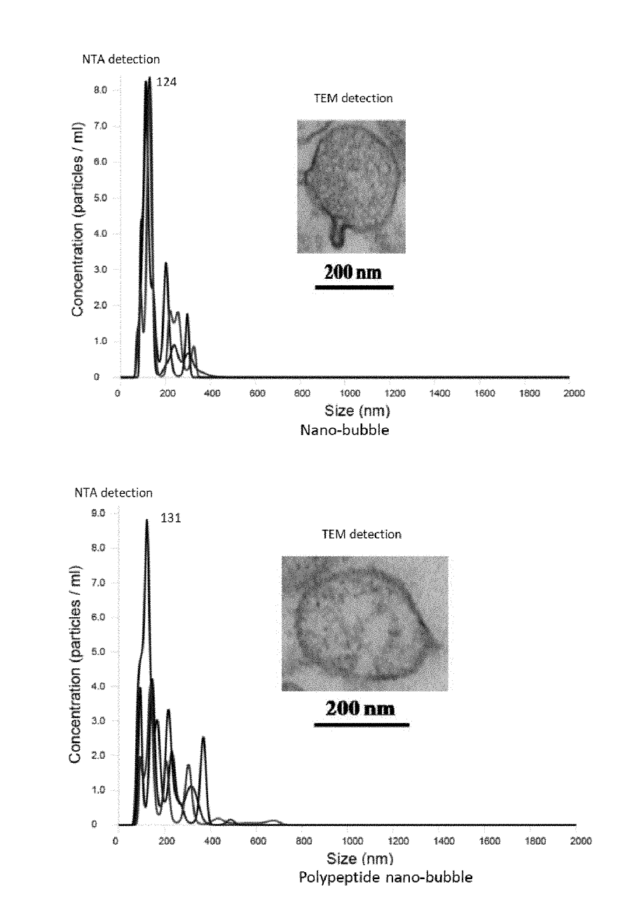

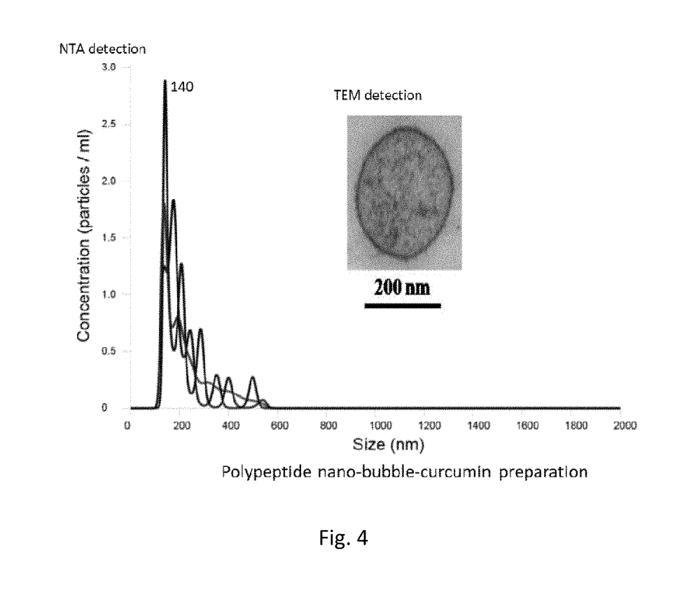

The culture solution of the 293T cells is high-glucose DMEM (Gibco, article number: 11995-065), and FBS (Gibco, article number: 10270-106) and penicillin/streptomycin double-antibody (Gibco, article number: 15140-122) are added, such that the final concentration of FBS is 10% (v/v), and the final concentration of the penicillin/streptomycin double-antibody is 1% (v/v).
3) Micro-bubbles, which are transfected with recombinant plasmids, of the 293T cells are extracted according to an ultracentrifugation method to obtain polypeptide modified nano-bubbles, namely polypeptide nano-bubbles. Specific steps are as follows: 48 h after the cells are transfected, the cell culture solution is collected, and 500 g of the cell culture solution is centrifuged for 15 min at 4° C.; supernatant is taken, cell precipitates are removed, and 1500 g of the supernatant is centrifuged for 20 min at 4° C.; supernatant is taken, and at the moment, precipitates are some cell debris or residual cell organelles; the supernatant is added in centrifugal tubes of an ultracentrifuge, balancing is carried out (difference between the two tubes which are balanced cannot exceed 0.2 g), and 110,000 g of the supernatant is centrifuged for 70 min at 4° C.; and the supernatant is abandoned. The precipitates are polypeptide modified nano-bubbles secreted by cells, are repeatedly blown and beaten to be dissolved with 100-200 μL PBS or a required empty culture solution, and are stored at 4° C.
4) Preparation of a polypeptide nano-bubble-curcumin preparation
   200 μg curcumin is mixed with 1 mg of the polypeptide nano-bubbles prepared in step (3), the mixture is cultured for 10 min at the room temperature, and then 200,000 g of the mixture is centrifuged for 90 min via sucrose density gradient (the concentrations of sucrose are separately 10%, 20%, 30%, 40%, 50%, 60% and 70%), and a yellow band is collected at 40-60% sucrose concentration, and is subjected to PBS resuspension to obtain the polypeptide nano-bubble-curcumin preparation.
   Simple process schematic diagrams of step 2), step 3) and step 4) are shown in FIG. 3.
5) Identification of targeting polypeptide modified nano-bubbles and polypeptide nano-bubble-curcumin preparation by TEM
   a. 2.5% of a glutaraldehyde fixing solution is separately added into polypeptide nano-bubble precipitates and polypeptide nano-bubble-curcumin precipitates, and fixing overnight is carried out at 4° C.;
   b. rinsing is carried out with PBS for three times, and every rinsing lasts for 10 min;
   c. a sample is fixed for 1 h with 1% osmium tetroxide at the room temperature, and the fixed sample is embedded with 10% gelatin;
   d. the sample is fixed for 1 h with glutaraldehyde in an environment of 4° C.;
   e. the sample is dehydrated with ethanol solutions of which the concentrations are increased (30%, 50%, 70%, 90%, 95%, 100%, 100% and 100%);
   f. after being soaked and embedded with epoxy resin, the sample is sliced with a Leica UC6 ultra-thin slicer; and
   g. the sample is observed with a transmission electron microscopy under the condition of 110 kV finally and is photographed, shown in FIG. 4.
6) NTA detection
   After the collected polypeptide nano-bubbles and polypeptide nano-bubble-curcumin are re-suspended, protein concentration (taking the concentration of the nano-bubbles as a guideline) is adjusted to 1 μg/mL, and the collected polypeptide nano-bubbles and polypeptide nano-bubble-curcumin are diluted by 100-500 times, and then are added in a Malvern Ns300 nano-particle counter for detection.
   The result is shown in FIG. 4, through TEM and NTA detection, it is discovered that all of the nano-bubbles, the polypeptide nano-bubbles and the polypeptide nano-bubble-curcumin preparation have clear lipid membrane structures, indicating that nano-bubbles before and after modification are obtained successfully. But along with addition of polypeptide and curcumin, the sizes of the nano-bubbles are obviously changed, indicating that the polypeptide nano-bubbles and the polypeptide nano-bubble-curcumin preparation are constructed successfully.

### Embodiment 2 The Polypeptide Nano-Bubble-Curcumin Preparation can Effectively Inhibit Increasing of Weight of Mice Fed with High Fat

Experimental animals: male mice at the age of 6 weeks and feed were provided by the Experimental Animal Center of Nanjing Medical University, license number: SCXK (Su) 2011-0003. The animals are fed in different cages randomly, and drink water freely.

Experimental drugs: normal diet control group (blank control group); and high-fat diet groups are divided into a normal saline group (model control group), a nano-bubble group, a nano-bubble-curcumin group, a polypeptide nano-bubble group (prepared by step 3) in embodiment 1), a curcumin group and a polypeptide nano-bubble-curcumin group (prepared by step 4) in embodiment 1).

Preparation steps of the nano-bubbles are as follows: 293T cells (ATCC, product number: CM-H010) are inoculated into a cell culture solution, the cell culture solution is cultured in a 5% CO2 culture tank at 37° C., the 293T cell culture solution which is not transfected is collected when the density reaches 70-90% (about 0.25-1×10⁶ cells), and 500 g of the 293T cell culture solution is centrifuged for 15 min at 4° C.; supernatant is taken, cell precipitates are removed, and 1500 g of the supernatant is centrifuged for 20 min at 4° C.; supernatant is taken, and at the moment, precipitates are some cell debris or residual cell organelles; the supernatant is added in centrifugal tubes of an ultracentrifuge, balancing is carried out (difference between the two tubes which are balanced cannot exceed 0.2 g), and 110,000 g of the supernatant is centrifuged for 70 min at 4° C.; and the supernatant is abandoned. The precipitates are nano-bubbles, are repeatedly blown and beaten to be dissolved with 100-200 μL PBS or a required empty culture solution, and are stored at 4° C.

Preparation steps of nano-bubble-curcumin are as follows: 200 μg curcumin is mixed with 1 mg of the prepared nano-bubbles, the mixture is cultured for 10 min at the room temperature, then 200,000 g of the mixture is centrifuged for 90 min via sucrose density gradient (concentrations of sucrose are separately 10%, 20%, 30%, 40%, 50%, 60% and 70%), and a yellow band is collected at 40-60% sucrose concentration, and is subjected to PBS resuspension to obtain the nano-bubble-curcumin preparation.

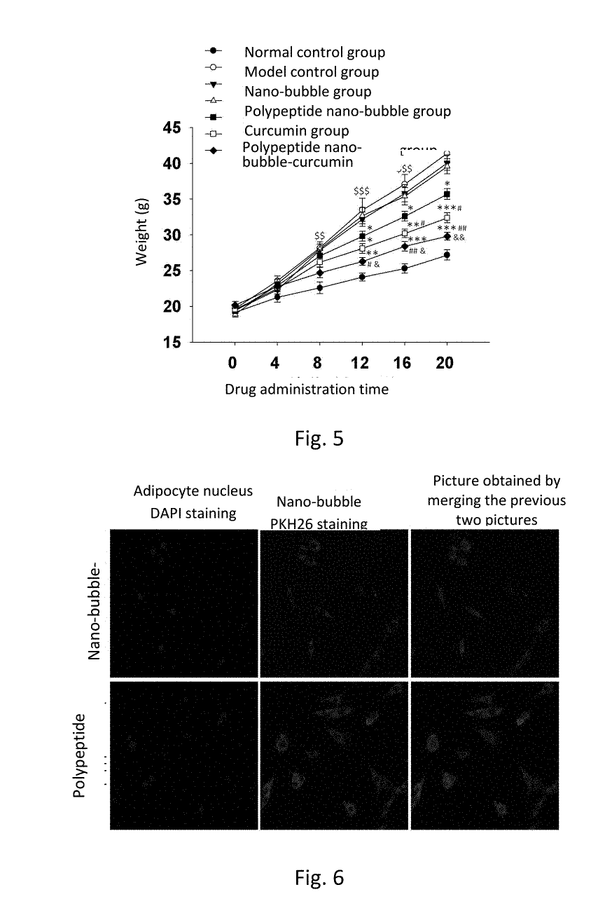

Experimental Steps:
1) 70 male mice at the age of 6 weeks are fed in a clean environment in the Experimental Animal Center of Nanjing Medical University at (21±2)° C. with the humidity being (35±2)%, are intermittently illuminated day and night 12 h:12 h, and take food and drink water freely, and drinking water is distilled water prepared by the Experimental Animal Center.
2) After the mice are adaptively fed for 2-3 days, 10 mice are randomly taken as mice in the normal control group and are fed with conventional feed, the rest 60 mice are grouped randomly, every group comprises 10 mice, and while fed with high-fat diet (1 wt. % of cholesterol, 20 wt. % of lard oil, 0.2 wt. % of bile salt and 78.8 wt. % of conventional feed), the mice are separately injected (illustrated by taking tail vein injection as an example) with normal saline (0.03 mL/30 g weight), nano-bubbles (40 μg/mice/time, dissolved in PBS), polypeptide nano-bubbles (taking dosage of the nano-bubbles as a guideline, 40 μg/mice/time, dissolved in PBS), curcumin (75 mg/kg weight, dissolved in PBS, the dosage is a low dosage according to the existing literatures), a nano-bubble-curcumin group (calculated by taking the dosage of the curcumin as a guideline, 75 mg/kg weight, dissolved in PBS) and polypeptide nano-bubble-curcumin (calculated by taking the dosage of the curcumin as a guideline, 75 mg/kg weight, dissolved in PBS) at 10 o'clock every morning. Expect for the mice in the normal control group, the mice in the other groups are continued fed with the high fat feed. The weight of the mice is weighed once every 4 weeks, drug administration is carried out for 20 weeks totally, and obtained data is subjected to statistical treatment. The result is shown in FIG. 5. From FIG. 5, we can see that from the eighth week during feeding, the weight increasing speed of the mice fed with the high fat feed (the model control group) is higher than the weight increasing speed of the mice fed normally (the normal control group), the difference has statistical significance (^{$$}p<0.01), indicating that the mice can be obese after being fed with high fat. In addition, while fed with the high-fat diet, the mice are separately injected with the nano-bubbles, the polypeptide nano-bubbles, the curcumin, the nano-bubble-curcumin and the polypeptide nano-bubble-curcumin, the result shows that compared with the mice in the model control group, increasing of the weight of the mice injected with the nano-bubbles and increasing of the weight of the mice injected with the polypeptide nano-bubbles are not inhibited obviously; 12 weeks after drug administration, compared with the model control group, increasing of the weight of the mice injected with the curcumin, the nano-bubble-curcumin and the polypeptide nano-bubble-curcumin are obviously inhibited, and the difference is remarkable (*p<0.05, **p<0.01, ***P<0.001). Importantly, along with prolonging of drug administration time, compared with the curcumin group, the weight increasing inhibiting effect of the nano-bubble-curcumin and the polypeptide nano-bubble-curcumin preparation are more obvious, and the difference is remarkable (^{#}P<0.05, ^{##}P<0.01). In addition, the weight reducing effect of the polypeptide nano-bubble-curcumin preparation is better than that of the nano-bubble-curcumin group (^{&}P<0.05, ^{&&}P<0.01), indicating that the weight increasing inhibiting effect of the polypeptide nano-bubble-curcumin preparation is more remarkable.

### Embodiment 3 The Polypeptide Nano-Bubble-Curcumin Preparation can Remarkably Inhibit Storage of Visceral Adipose and Subcutaneous Adipose of Mice Fed with High Fat

1) The obese mice fed with high fat are subjected to drug administration by intravenous injection for 20 weeks according to experimental steps in embodiment 2;
2) the mice in the various groups are not fed with food for 12 hours, and are weighed accurately after being anaesthetized with 3% pentobarbital sodium at 45 mg/kg by intraperitoneal injection;
3) the mice are conventionally sterilized and subjected to laparotomy, kidney surrounding adipose tissues, mesenterium surrounding adipose tissues, epididymis surrounding adipose tissues and abdominal subcutaneous adipose tissues are rapidly picked to weigh wet weight (wherein the range of the abdominal subcutaneous adipose tissues is as follows: the abdomen below an costal arch and above a groin, and a midaxillary line is taken as a boundary for two sides);
4) the weight is calculated according to the following formula: wet weight of abdominal adipose; adipose pad total weight (g)=the kidney surrounding adipose tissues+the epididymis surrounding adipose tissues; and body adipose content=the adipose pad total weight (g)/body weight (g).
5) The obtained data is subjected to statistical treatment. The results are shown in Table 6, Table 7 and Table 8.

TABLE 6
Inhibition effect of polypeptide nano-bubble-curcumin preparation to storage of visceral adipose of mice n = 10

| | Amount of visceral adipose tissues (g) | | | |
|---|---|---|---|---|
| | | | Omentum and | Total weight of |
| | Epididymis | Kidney | mesenterium | visceral |
| Group | surrounding | surrounding | surrounding | adipose tissues |
| Normal control group | 0.50 ± 0.13 | 0.09 ± 0.03 | 0.21 ± 0.02 | 0.75 ± 0.13 |
| Model control group | 2.51 ± 0.14^{$$$} | 1.06 ± 0.11^{$$$} | 0.73 ± 0.09^{$$$} | 10.01 ± 0.24^{$$$} |
| Nano-bubble group | 2.49 ± 0.11 | 1.05 ± 0.07 | 0.81 ± 0.06 | 9.91 ± 0.21 |
| Polypeptide nano- | 2.54 ± 0.1 | 1.08 ± 0.06 | 0.79 ± 0.07 | 10.03 ± 0.19 |
| bubble group | | | | |
| Curcumin group | 1.21 ± 0.18* | 0.72 ± 0.05* | 0.59 ± 0.02* | 8.03 ± 0.02* |
| Nano-bubble-curcumin | 0.96 ± 0.12**^{#} | 0.58 ± 0.02**^{#} | 0.47 ± 0.01**^{#} | 6.14 ± 0.04**^{#} |
| group | | | | |
| Polypeptide nano- | 0.67 ± 0.05***^{##&&} | 0.31 ± 0.01***^{##&&} | 0.33 ± 0.02***^{##&&} | 3.36 ± 0.03***^{##&&} |
| bubble-curcumin group | | | | |

Note:
^{$$$}P < 0.001 shows comparison with the normal control group;
*P < 0.05,
**P < 0.01 and
***P < 0.001 show comparison with the model control group;
^{#}P < 0.05 and
^{##}P < 0.01 show comparison with the curcumin group; and
^{&&}P < 0.01 shows comparison with the nano-bubble-curcumin group.

TABLE 7
Inhibition effect of polypeptide nano-bubble-curcumin preparation to body fat mass index of mice n = 10

| Group | Fat pad total weight (g) | Weight (g) | Body fat mass index (%) |
|---|---|---|---|
| Normal control group | 0.53 ± 0.11 | 28.58 ± 0.95 | 1.68 ± 0.29 |
| Model control group | 3.65 ± 0.31^{$$$} | 42.42 ± 1.32^{$$$} | 8.98 ± 0.35^{$$$} |
| Nano-bubble group | 3.43 ± 0.12 | 40.12 ± 0.49 | 8.87 ± 0.22 |
| Polypeptide nano-bubble | 3.55 ± 0.25 | 41.61 ± 0.75 | 9.01 ± 0.17 |
| group | | | |
| Curcumin group | 2.34 ± 0.10* | 34.92 ± 0.96* | 6.38 ± 0.21* |
| Nano-bubble-curcumin group | 1.67 ± 0.10**^{#} | 32.01 ± 0.32**^{#} | 4.13 ± 0.14**^{#} |
| Polypeptide nano-bubble- | 1.01 ± 0.05***^{##&&} | 30.75 ± 0.24***^{##&&} | 2.08 ± 0.11***^{##&&} |
| curcumin group | | | |

Note:
^{$$$}P < 0.001 shows comparison with the normal control group;
*P < 0.05,
**P < 0.01 and
***P < 0.001 show comparison with the model control group;
^{#}P < 0.05 and
^{##}P < 0.01 show comparison with the curcumin group; and
^{&&}P < 0.01 shows comparison with the nano-bubble-curcumin group. The fat pad total weight = kidney surrounding adipose tissues + epididymis surrounding adipose tissues; and the body fat mass index = fat pad total weight (g)/weight (g).

TABLE 8
Inhibition effect of polypeptide nano-bubble-curcumin preparation to storage of subcutaneous adipose of mice n = 10

| Group | SCAT (g) | VAT (g) | Weight (g) | SCAT/weight | VAT/SCAT |
|---|---|---|---|---|---|
| Normal control group | 0.52 ± 0.13 | 0.75 ± 0.13 | 27.2 ± 0.73 | 1.63 ± 0.31 | 0.84 ± 0.16 |
| Model control group | 4.13 ± 0.24^{$$$} | 10.01 ± 0.24^{$$$} | 41.42 ± 1.31^{$$$} | 9.74 ± 0.37^{$$$} | 2.97 ± 0.13^{$$$} |
| Nano-bubble group | 4.07 ± 0.12 | 9.91 ± 0.21 | 40.12 ± 1.07 | 9.46 ± 0.29 | 2.91 ± 0.09 |
| Polypeptide nano-bubble | 4.15 ± 0.21 | 10.03 ± 0.19 | 39.6 ± 1.04 | 9.58 ± 0.31 | 2.88 ± 0.11 |
| group | | | | | |
| Curcumin group | 3.01 ± 0.05* | 7.24 ± 0.02* | 35.72 ± 0.75* | 7.02 ± 0.38* | 1.78 ± 0.09* |
| Nano-bubble-curcumin | 2.13 ± 0.05**^{#} | 5.36 ± 0.02**^{#} | 32.44 ± 0.7**^{#} | 5.16 ± 0.38**^{#} | 1.41 ± 0.02**^{#} |
| group | | | | | |
| Polypeptide | 1.01 ± 0.03***^{##&&} | 2.32 ± 0.03***^{##&&} | 29.75 ± 0.54***^{##&&} | 3.98 ± 0.20***^{##&&} | 1.03 ± 0.03***^{##&&} |
| nano-bubble-curcumin | | | | | |
| group | | | | | |

Note:
^{$$$}P < 0.001 shows comparison with the normal control group;
*P < 0.05,
**P < 0.01 and
***P < 0.001 show comparison with the model control group,
^{#}P < 0.05 and
^{##}P < 0.01 show comparison with the curcumin group; and
^{&&}P < 0.01 shows comparison with the nano-bubble-curcumin group.
SCAT: subcutaneous adipose tissue total weight; and
VAT: total weight of visceral adipose tissues.

From Table 6, we can see that the visceral adipose of the mice fed with the high-fat diet in the model control group, such as epididymis fat, kidney surrounding adipose and omentum and mesenterium surrounding fat, is obviously increased than that of the mice in the normal control group, and the difference has statistical significance (^{$$$}P<0.001), indicating that obesity causes storage of a large number of visceral fat. Compared with the model control group, after the mice are injected with the nano-bubbles and the polypeptide nano-bubbles, the wet weight of the visceral adipose tissues of the mice is not reduced obviously. But after the mice are treated with the curcumin, the nano-bubble-curcumin and the polypeptide nano-bubble-curcumin preparation, the wet weight of the visceral adipose tissues of the mice is reduced obviously, compared with the model control group, the difference is remarkable (*P<0.05, **P<0.01, ***P<0.001), indicating that the curcumin, the nano-bubble-curcumin and the polypeptide nano-bubble-curcumin preparation obviously inhibit storage of the visceral adipose of the mice fed with high fat. Compared with the curcumin group, the inhibiting effects of the nano-bubble-curcumin and the polypeptide nano-bubble-curcumin preparation to storage of the visceral adipose of the mice are more obvious, and the difference is remarkable (^{#}P<0.05, ^{##}P<0.01). In addition, the inhibiting effect of the polypeptide nano-bubble-curcumin preparation to storage of the visceral adipose of the mice is higher than that of the nano-bubble-curcumin group (^{&&}P<0.01), indicating that the inhibiting effect of the polypeptide nano-bubble-curcumin preparation to increasing of the visceral adipose of the mice fed with high fat is more remarkable.

Change of the body fat mass indexes (F-IDX) of the mice fed with high fat before and after the mice are subjected to administration of the nano-bubbles, the polypeptide nano-bubbles, the curcumin, the nano-bubble-curcumin and the polypeptide nano-bubble-curcumin preparation is further detected, and the result is shown in Table 7, and the fat pad total weight and the body fat mass index of the mice in the model control group are obviously higher than those of the mice in the normal control group (^{$$$}P<0.001). Compared with the model control group, after the mice are injected with the nano-bubbles and the polypeptide nano-bubbles, the body fat mass indexes of the mice are not improved. But after the mice are treated with the curcumin, the nano-bubble-curcumin and the polypeptide nano-bubble-curcumin preparation by tail vein injection, the body fat mass index of the mice are obviously reduced, the difference is obvious (*P<0.05, **P<0.01, ***P<0.001), further indicating that the curcumin, the nano-bubble-curcumin and the polypeptide nano-bubble-curcumin preparation can remarkably inhibit increasing of the visceral adipose of the mice fed with high fat. Compared with the curcumin group, the body fat mass index reducing degree of the mice treated with the nano-bubble-curcumin and the polypeptide nano-bubble-curcumin preparation is more obvious, and the difference is remarkable (^{#}P<0.05, ^{##}P<0.01). In addition, the body fat mass index reducing effect of the polypeptide nano-bubble-curcumin preparation is higher than that of the nano-bubble-curcumin group (^{&&}P<0.01), indicating that the inhibiting effect of the polypeptide nano-bubble-curcumin preparation to increasing of the body fat mass index of the mice fed with high fat is more remarkable.

As shown in Table 8, compared with the curcumin group, storage of the visceral adipose of the mice fed with high fat is obviously reduced by treatment with the nano-bubble-curcumin and the polypeptide nano-bubble-curcumin preparation, furthermore, the weight of the subcutaneous adipose of the mice fed with high fat is reduced remarkably, the ratio of the subcutaneous adipose accounting for the weight is reduced more obviously, and the difference is remarkable (^{#}P<0.05, ^{##}P<0.01). Importantly, compared with the nano-bubble-curcumin group, the polypeptide nano-bubble-curcumin treatment group can effectively inhibit storage of the subcutaneous adipose of the mice (^{&&}P<0.01). But the weight of the subcutaneous adipose of the mice injected with the nano-bubbles and polypeptide-nano-bubbles is not reduced remarkably. The foregoing results all prompt that compared with the normal control group, the abdominal visceral adipose and the abdominal subcutaneous adipose of the mice fed with high fat are obviously increased, after the mice are treated with the curcumin, the nano-bubble-curcumin and the polypeptide nano-bubble-curcumin preparation, compared with the model group, the wet weight of the abdominal visceral adipose and the abdominal subcutaneous adipose of the mice fed with high fat are remarkably reduced, and compared with the nano-bubble-curcumin group, the inhibiting effect of the polypeptide nano-bubble-curcumin preparation is better. In combination with the results of Embodiment 2, it is further indicated that the polypeptide nano-bubble-curcumin preparation further has an effect of inhibiting obesity remarkably.

### Embodiment 4 The Polypeptide Nano-Bubble-Curcumin Preparation Effectively Facilitates the Curcumin to Enter Adipocytes

1) Red fluorescent dye PKH26 labeled (for labeling nano-bubbles) nano-bubble-curcumin and polypeptide nano-bubble-curcumin are added in adipocytes 3T3-L1, and states of the red fluorescent dye PKH26 labeled nano-bubble-curcumin and the polypeptide nano-bubble-curcumin which enter the cells are observed by a laser scanning confocal microscope. Specific steps are as follows: 1) 25 μL of 2×10⁷ PBS dissolved nano-bubble-curcumin suspension and 25 μL of 2×10⁷ PBS dissolved polypeptide nano-bubble-curcumin suspension are separately taken, and separately added into diluent C of 1 mL PKH26 dye liquor (Shanghai Beinuo Biotech Co., Ltd., article number: MINI26-1KT) for re-suspending; 2) PKH26 ethanol dye liquor is diluted with the diluent C until the final concentration reaches 2 μM and the final volumes are 1 mL separately; 3) 1 mL of the diluted nano-bubble-curcumin suspension and 1 mL of the diluted polypeptide nano-bubble-curcumin suspension are added in the 1 mL PKH26 dye liquor as soon as possible, a sample is rapidly mixed uniformly with a straw immediately, and is incubated at the room temperature (20-25° C.) for 2-5 min, and a centrifugal tube is slightly inverted on time to ensure full uniform mixing; 4) the same amount of serum or 1% BSA is added to incubate for 1 min to stop staining reaction, the same amount of PBS containing serum is used to dilute terminated reaction liquid, 100,000-160,000 g of the liquid is centrifuged for 1-2 h at 20-25° C., and supernatant is removed; and 5) a precipitate mass is transferred into a new centrifugal tube, and is further washed for three times, then the precipitate is re-suspended with 50-100 μL PBS, and is added in 2 mL of a culture solution which cultures 3T3-L1 cells, and the culture solution is cultured in a 5% CO2 culture tank at 37° C. for 4-24 h.
2) The states of red labeled nano-bubble-curcumin and polypeptide nano-bubble-curcumin which enter the adipocytes are observed by a laser scanning confocal microscope.
   The result is shown in FIG. 6 (400×), compared with the adipocytes treated with the nano-bubble-curcumin, the adipocytes treated with the red fluorescence labeled polypeptide nano-bubble-curcumin preparation is higher in red fluorescence intensity, indicating that the polypeptide nano-bubble-curcumin preparation can efficiently enter the adipocytes, namely the polypeptide nano-bubbles can effectively transfer the curcumin to target cells-adipocytes. In combination with animal experimental data in Embodiment 2 and Embodiment 3, it further prompts that compared with the nano-bubble-curcumin group, the curcumin can more efficiently enter the target cells-adipocytes to play functions under the targeting effect of the polypeptide nano-bubbles, and thus, obesity can be inhibited more effectively.

### Embodiment 5 The Polypeptide Nano-Bubble-Curcumin Preparation does not have Obvious Biological Toxicity

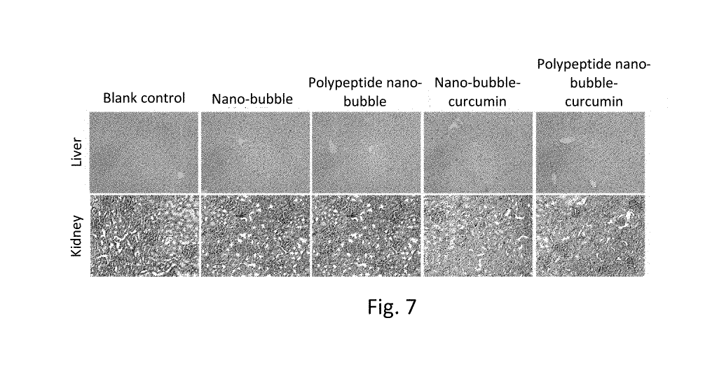

1) According to experimental steps of embodiment 2, the mice fed normally are subjected to drug administration by intravenous injection for 20 weeks, the mice in a blank control group are injected with normal saline (0.03 ml/30 g weight), and the mice in the experimental group are separately injected with the nano-bubbles (80 μg/mice/time), the polypeptide nano-bubbles (calculated by taking the dosage of the nano-bubbles as a guideline, 80 μg/mice/time), the nano-bubble-curcumin and the polypeptide nano-bubble-curcumin preparation (calculated by taking the dosage of the curcumin as a guideline, 150 mg/kg weight).
2) The mice in various groups are not fed with food for 12 hours, and are conventionally sterilized for laparotomy after being anaesthetized with 3% pentobarbital sodium at 45 mg/kg by intraperitoneal injection, liver and kidney tissues are rapidly picked for H-E staining observation, and the obtained result is shown in FIG. 7.
   From FIG. 7 (200×), we can see that compared with the blank control group, the liver and kidney tissues of the mice treated with the nano-bubbles, the polypeptide nano-bubbles, the nano-bubble-curcumin and the polypeptide nano-bubble-curcumin preparation by tail vein injection do not have obvious pathologic change, indicating that the nano-bubbles, the polypeptide nano-bubbles, the nano-bubble-curcumin and the polypeptide nano-bubble-curcumin preparation do not have obvious toxic and side effects on the mice.

What is claimed is:

1. A preparation method for polypeptide nano-bubbles, characterized by comprising the following steps:
(1) constructing a recombinant plasmid, the recombinant plasmid comprising a Flag tag, an adipose tissue-targeting polypeptide and the coding gene of micro-bubble marker membrane protein CD63, wherein the Flag tag and the adipose tissue-targeting polypeptide are successively linked to the rear of the start codon of the coding gene of the micro-bubble marker membrane protein CD63, and the amino acid sequence of the adipose tissue-targeting polypeptides is shown as SEQ ID NO: 8; and
(2) transfecting the recombinant plasmid obtained in step (1) into cells which secrete nano-bubbles through lipidosome, culturing to collect cell culture solution, and extracting the polypeptide nano-bubbles by ultracentrifugation.

2. The preparation method according to claim 1, characterized in that steps of construction of the recombinant plasmid in step (1) are as follows: amplifying Flag-peptide-CD63 fragment by PCR using cDNA of CD63 as template and CD63-F/CD63-R as primers, and inserting the Flag-peptide-CD63 fragment into Xhol and EcoRI digestion sites of carrier plasmid pIRES2-EGFP to obtain the recombinant plasmid, wherein the nucleotide sequence of the primer CD63-F is shown in SEQ ID NO: 1, and the nucleotide sequence of the primer CD63-R is shown in SEQ ID NO: 2.

3. The preparation method according to claim 1, characterized in that the cells which secrete the nano-bubbles in step (2) are immature dendritic cells or mesenchymal stem cells.

4. The preparation method according to claim 1, characterized in that the cells which secrete the nano-bubbles in step (2) are 293T cells.

5. The preparation method according to claim 1, characterized in that steps of the ultracentrifugation in step (2) are as follows: centrifuging the cell culture solution at 500-1000 g, 2-4° C. for 10-30 min, and then taking first-time supernatant; centrifuging the first-time supernatant at 1500-3000 g, 2-4° C. for 20-30 min, and then taking second-time supernatant; and centrifuging the second-time supernatant at 100,000-160,000 g, 2-4° C. for 1-2 h to obtain precipitates, namely the polypeptide nano-bubbles.

6. A polypeptide nano-bubbles prepared by the preparation method of claim 1.

7. An application of the polypeptide nano-bubbles of claim 6 as a drug carrier in preparation of drugs for treating obesity.

8. The application according to claim 7, characterized in that the drugs for treating obesity are nucleic acids drugs, proteins drugs, lipids drugs, carbohydrates drugs or ketones drugs.

9. The application according to claim 8, characterized in that the ketones drugs are curcumin.
